(19) United States
(12) Patent Application Publication
(10) Pub. No.: US 2024/0148432 A1
(43) Pub. Date: May 9, 2024

(54) FLUID-ENHANCED ELECTROSURGERY WITH INTEGRATED IRRIGATION AND ASPIRATION

(71) Applicant: Medtronic Advanced Energy LLC, Minneapolis, MN (US)

(72) Inventors: Xiaoming CHENG, Marana, AZ (US); Drew M. WILSON, Boulder, CO (US); Matthew S. PALM, Black Hawk, CO (US); Yahia LAOUAR, Superior, CO (US); Yeung CHOW, Longmont, CO (US)

(21) Appl. No.: 18/500,809

(22) Filed: Nov. 2, 2023

Related U.S. Application Data
(60) Provisional application No. 63/422,401, filed on Nov. 3, 2022.

(51) Int. Cl. A61B 18/14

(52) U.S. Cl. CPC A61B 18/1482; A61B 2018/00577; A61B 2218/002; A61B 2218/007

(57) ABSTRACT

An electrosurgical device includes a distal portion having a first electrode extending distally from an elongated shaft, wherein the first electrode is configured to provide a delivered electrical current to a target treatment site within a patient, and wherein the first electrode defines a first irrigation port configured to release a surgical fluid into the target treatment site; and a second electrode extending distally from the elongated shaft, wherein the second electrode is configured to receive a return electrical current from the target treatment site, and wherein the second electrode defines a second irrigation port configured to release the surgical fluid into the target treatment site; wherein the distal portion of the electrosurgical device defines at least one aspiration port configured to proximally aspirate the surgical fluid from the target treatment site.

### CROSS-REFERENCE TO RELATED APPLICATIONS

This application claims the benefit of Provisional Application No. 63/422,401, filed Nov. 3, 2022, the disclosure of which is incorporated by reference herein.

### FIELD

The present disclosure relates to electrosurgery.

### BACKGROUND

Electrosurgical devices for applying electrical energy to tissue may be used in surgical procedures for hemostatic sealing or coagulation of soft tissue and bone at the operative site. Such electrosurgical devices can be used for, but not limited to orthopedic, spine, thoracic, or open abdominal surgery.

An electrosurgical device may include a handheld unit having a distal end with one or more electrodes. The one or more electrodes can be positioned proximate the target tissue such that an electrical current is introduced into the tissue. The resulting generated heat can be used to cut, coagulate, or induce metabolic processes in the target tissue. The electrosurgical device can be used with an electrosurgical generator which generally provides power and electrical energy in the form of radio frequency (“RF”) energy via either of two handpiece topologies (or a particular combination thereof): monopolar or bipolar.

During monopolar operation, an active electrode introduces current into the target tissue. The current returns through a return electrode separately located on a patient's body. Accordingly, the monopolar handpiece has only one wire for the treatment signal in the monopolar connector—the second contact, known as the “return signal” exists in a different connector known as a “return-pad connector.” During bipolar operation, current is introduced into, and returned from, the target tissue via “active” and “return” electrodes located on the bipolar handpiece.

Conventional electrosurgical devices used for electrosurgical tissue treatment face an array of challenges that can vary across procedures. Some challenges that can arise are the use of multiple different devices to perform individual functions, thereby both complicating the procedure and occupying a greater amount of a limited space, both internal to the patient and within the operating environment.

### SUMMARY

The techniques of this disclosure generally relate to a handheld electrosurgical device configured to: irrigate, disperse, or infuse a surgical fluid (e.g., saline); ablate or cauterize tissue in the presence of the fluid; and simultaneously or subsequently aspirate the residual fluid from the target treatment site.

In one aspect, the present disclosure provides an electrosurgical device having a proximal portion including an electrical connector configured to electrically couple to a generator configured to provide electrical energy, and a distal portion. The distal portion includes a first electrode extending distally from an elongated shaft, wherein the first electrode is configured to provide a delivered electrical current to a target treatment site within a patient, and wherein the first electrode defines a first irrigation port configured to release a surgical fluid into the target treatment site. The distal portion further includes a second electrode extending distally from the elongated shaft, wherein the second electrode is configured to receive a return electrical current from the target treatment site, and wherein the second electrode defines a second irrigation port configured to release the surgical fluid into the target treatment site. The distal portion of the electrosurgical device defines at least one aspiration port configured to proximally aspirate the surgical fluid from the target treatment site.

In another aspect, the present disclosure provides a method of performing electrosurgery, the method comprising delivering, via a first irrigation port defined by a first electrode of a distal portion of an electrosurgical device and via a second irrigation port defined by a second electrode of the distal portion of the electrosurgical device, a surgical fluid into a target treatment site within a patient. The method further comprises providing via the first electrode, a delivered electrical current to the target treatment site, receiving via the second electrode, a return electrical current from the target treatment site, and aspirating via an aspiration port defined by the distal portion of the electrosurgical device, the surgical fluid from the target treatment site.

In another aspect, the present disclosure provides a medical system including a generator configured to provide electrical energy, and an electrosurgical device. The electrosurgical device includes a proximal portion having an electrical connector configured to electrically couple to the generator, and a distal portion having a first electrode extending distally from an elongated shaft, wherein the first electrode is configured to provide a delivered electrical current to a target treatment site within a patient, and wherein the first electrode defines a first irrigation port configured to release a surgical fluid into the target treatment site. The distal portion further includes a second electrode extending distally from the elongated shaft, wherein the second electrode is configured to receive a return electrical current from the target treatment site, and wherein the second electrode defines a second irrigation port configured to release the surgical fluid into the target treatment site. The distal portion of the electrosurgical device defines at least one aspiration port configured to proximally aspirate the surgical fluid from the target treatment site.

In another aspect, the present disclosure provides techniques for using a handheld electrosurgical device to perform an electrosurgical procedure, including both irrigating and aspirating a surgical fluid via the handheld devices described herein.

Examples of the present disclosure advantageously reduce the number of surgical tools required in the field, allowing irrigation and aspiration of surgical fluid to be performed by the same tool providing the electrosurgery.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Subject matter hereof may be more completely understood in consideration of the following detailed description of various examples in connection with the accompanying figures, in which:

FIG. 1 is a front view of an example medical system having an electrosurgical generator unit, a surgical-fluid source, and a handheld electrosurgical device;

FIG. 2 is a front perspective view of the electrosurgical generator unit of FIG. 1,

FIG. 3 is a rear view of the electrosurgical generator unit of FIG. 1;

FIG. 4 is a perspective view of an electrosurgical device according to the present invention;

FIG. 5 is a close-up cross-sectional view of a distal portion of the electrosurgical device of FIGS. 1 and 4 with an exemplary fluid coupling to a tissue surface of a patient's tissue at a target treatment site;

FIG. 6 is a perspective view of the distal portion of the handheld electrosurgical device of FIG. 5;

FIG. 7 is an under-side view of the distal portion of the handheld electrosurgical device of FIG. 5;

FIG. 8 is a flowchart illustrating a technique for performing an electrosurgical procedure via the example electrosurgical devices described herein.

### DETAILED DESCRIPTION

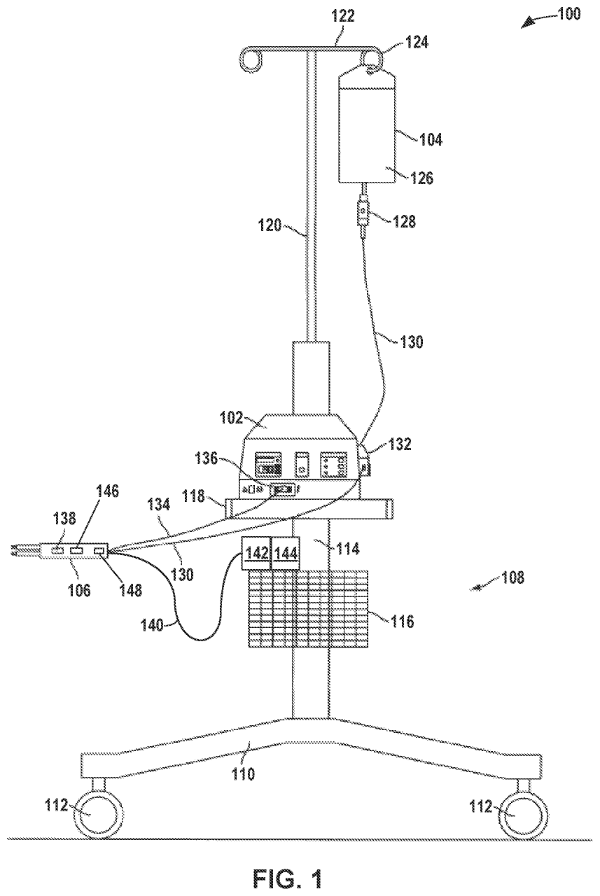

FIG. 1 depicts an example medical system 100 having an electrosurgical generator unit 102 in combination with a fluid source 104 and a handheld electrosurgical device 106. Certain elements of medical system 100 are detailed further in commonly assigned U.S. Pat. No. 8,882,756, entitled “FLUID-ASSISTED ELECTROSURGICAL DEVICES, METHODS AND SYSTEMS,” the entire contents of which are incorporated by reference herein.

The example of system 100 shown in FIG. 1 includes a movable cart 108 having a chassis 110 which is provided with two or more wheels 112 for easy transportation. The chassis 110 carries a support member 114 including a hollow cylindrical post to which a storage basket 116 may be fastened and used to store a user manual for electrosurgical unit 102, as well as additional unused devices. Furthermore, the support member 114 carries a platform 118 (e.g., a pedestal table) to provide a flat, stable surface for retaining electrosurgical unit 102.

As shown in FIG. 1, cart 108 further includes a surgical-fluid-source-carrying pole 120 having a height that may be adjusted by sliding the carrying pole 120 up and down within the support member 114, and thereafter securing the pole 120 in position with a set screw (not shown). On the top of the fluid-source-carrying pole 120 is a cross support 122 provided with loops 124 at the ends thereof to provide a hook for carrying surgical-fluid source 104.

As shown in FIG. 1, fluid source 104 includes a bag of surgical fluid (e.g., saline) from which the fluid 126 flows through a drip chamber 128 after the bag is penetrated with a spike disposed at the end of the drip chamber 128. Thereafter, surgical fluid 126 flows through flexible delivery tubing 130 to handheld electrosurgical device 106. The fluid delivery tubing 130 can be formed from a polymer material.

As shown in FIG. 1, the fluid delivery tubing 130 passes through pump 132. In the example shown in FIG. 1, pump 132 includes a peristaltic pump and, more specifically, a rotary peristaltic pump. With a rotary peristaltic pump, a portion of the delivery tubing 130 is loaded into the pump head by raising and lower the pump head in a predetermined manner. Surgical fluid 126 is conveyed within the delivery tubing 130 by waves of contraction, directed externally onto the tubing 130, which are produced mechanically, typically by rotating pinch rollers that rotate on a driveshaft to intermittently compress the tubing 130 against an anvil support. Additionally or alternatively, pump 132 can include a linear peristaltic pump. With a linear peristaltic pump, surgical fluid 126 is conveyed within the delivery tubing 130 by waves of contraction, directed externally onto the tubing 130, which are produced mechanically, typically by a series of compression fingers or pads which sequentially squeeze the tubing 130 against a support.

In some examples, the surgical fluid 126 includes saline, preferably normal (physiologic) saline, however, any other suitable electrically conductive fluids may be used instead or in addition. While a conductive fluid is preferred, surgical fluid 126 can also include a non-conductive (e.g., electrically insulative) fluid. The use of a non-conductive fluid is less preferred than a conductive fluid, however, the use of a non-conductive fluid still provides certain advantages over the use of dry electrodes including, for example, reduced occurrence of tissue adhering to electrodes of handheld device 106 and cooling of the electrodes and/or tissue. Therefore, it is also within the scope of the present disclosure to include the use of a non-conducting fluid, such as deionized water.

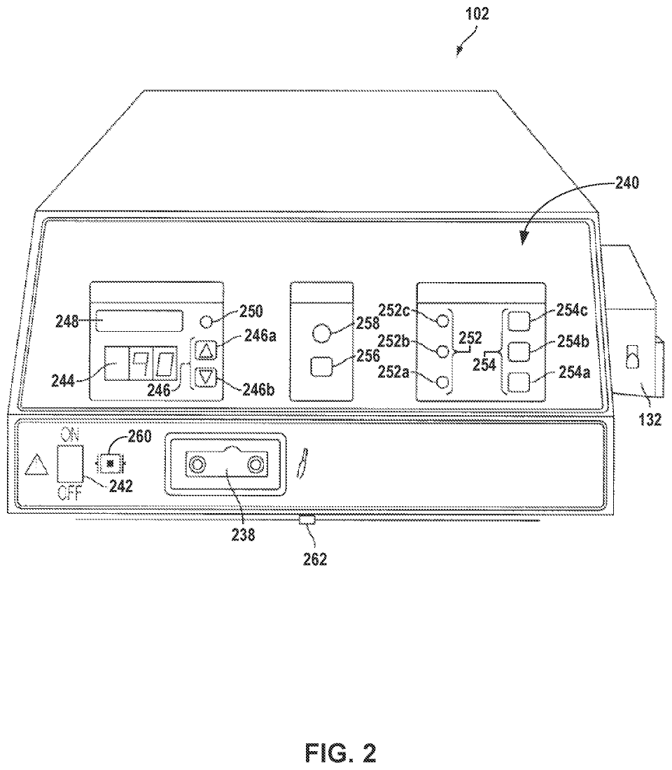

As shown in FIG. 1, handheld electrosurgical device 106 is electrically coupled, via cable 134, to electrosurgical unit 102, which includes a plurality of electrically insulated wire conductors and at least one plug 136 at the end thereof. The electrosurgical unit 102 provides radio-frequency (RF) energy via cable 134 to handheld electrosurgical device 106. As shown in FIG. 2, plug receptacle 238 of electrosurgical unit 102 receives the plug 136 of device 106 therein to electrically connect device 106 to the electrosurgical unit 102. The fluid-delivery tubing 130 can be integrated with cable 134 and produced with the electrically insulated wires via plastic co-extrusion.

In accordance with techniques of this disclosure, handheld electrosurgical device 106 is configured to both irrigate surgical fluid 126 into the target treatment site, and also subsequently aspirate residual surgical fluid 126 from the target treatment site. In this way, electrosurgical device 106 is configured to reduce a complexity of the surgical procedure, and also to reduce a net form factor of the set of surgical devices required to complete the procedure, thereby further improving patient outcomes.

For instance, as shown in FIG. 1, handheld device 106 may be fluidically coupled, via flexible fluid-withdrawal tubing 140, to a suction source 142 and a discharge reservoir 144. Handheld device 106 can include various user-input mechanisms, such as buttons, switches, levers, triggers, toggles, knobs, or the like, configured to control irrigation and aspiration of surgical fluid 126 via a distal portion of handheld device 106. For instance, the clinician may actuate a first user-input mechanism 146 to deploy surgical fluid 126 into the target treatment site, and can actuate another user-input mechanism 148 to actuate suction source 142 to aspirate the surgical fluid from the target treatment site. In some examples the user-input mechanisms (or additional user-input mechanisms) are configured to enable the user to control a rate or intensity of irrigation and/or aspiration, as appropriate. For instance, a control knob on handheld device 106 may be configured to increase or decrease an amount of suction force applied by suction source 142 independently of other system parameters. In some examples, suction source 142 can include a suction source provided by the facility in which the electrosurgical procedure is occurring (e.g., hospital or other care center).

FIG. 2 shows an example front panel 240 of the electrosurgical unit 102 (or “generator unit” 102) of FIG. 1. Front panel 240 includes a power switch 242 configured to turn the electrosurgical unit 102 on and off. After turning the electrosurgical unit 102 on, the RF-power-setting display 244 is used to display the RF power setting numerically in watts. In some examples, the power-setting display 244 includes a liquid crystal display (LCD), or other suitable display screen. Additionally, this display 244 is used to display errors, in which case the display 244 can indicate “Err” along with relevant error-code number(s).

The RF power selector 246 includes RF-power-setting switches 246a, 246b, which are used to select the RF power setting. Pushing switch 246a increases the RF power setting, while pushing switch 246b decreases the RF power setting. RF power output may be set in 5-watt increments in the range of 20 to 100 watts, and 10-watt increments in the range of 100 to 200 watts. Additionally, electrosurgical unit 102 includes an RF-power-activation display 248 including an indicator light 250 which illuminates when RF power is activated. Switches 246a, 246b can include membrane switches.

In addition to RF-power-setting display 244, electrosurgical unit 102 further includes a fluid-flow-rate-setting display 252. Flow-rate-setting display 252 includes three indicator lights 252a, 252b 252c, with first light 252a corresponding to a fluid-flow-rate setting of “low,” second light 252b corresponding to a fluid-flow-rate setting of “medium” (or “intermediate”), and third light 252c corresponding to a flow-rate setting of “high.” One of these three indicator lights 252 will illuminate when the corresponding fluid-flow-rate setting is selected.

A fluid-flow selector 254, including flow-rate setting switches 254a, 254b, 254c, is used to select or switch the flow-rate setting. Three push switches 254 are provided, with first switch 254a corresponding to a fluid-flow-rate setting of “low,” second switch 254b corresponding to a fluid-flow-rate setting of “medium” (or “intermediate”), and third switch 254c corresponding to a flow-rate setting of “high.” Pushing one of these three switches 254 selects the corresponding flow-rate setting of either “low,” “medium” (“intermediate”), or “high.” The “medium,” or “intermediate,” flow-rate setting is automatically selected as the default setting if no other setting is manually selected. Switches 254a, 254b, and 254c can include membrane switches.

Before commencing an electrosurgical procedure, it may be desirable to prime handheld device 106 (FIG. 1) with surgical fluid 126. Priming is desirable to inhibit RF power activation without the presence of fluid 126. Accordingly, a priming switch 256 (FIG. 2) is used to initiate priming of handheld device 106 with surgical fluid 126. Pushing switch 256 one time initiates operation of pump 132 for a predetermined time duration in order to prime handheld device 106. After expiration of the predetermined time duration, the pump 132 shuts off automatically. When priming of handheld device 106 is initiated, a priming display 258 (e.g., an indicator light) illuminates during the priming cycle.

On the front panel 240, a bipolar activation indicator 260 illuminates when RF power is activated from the electrosurgical unit 102, either via switch 138 (FIG. 1) on handheld device 106 or via a footswitch (not shown). A pullout drawer 262 (FIG. 2) is located under the electrosurgical unit 102 where the user (e.g., a clinician) of electrosurgical unit 102 may find a short form of the user's manual.

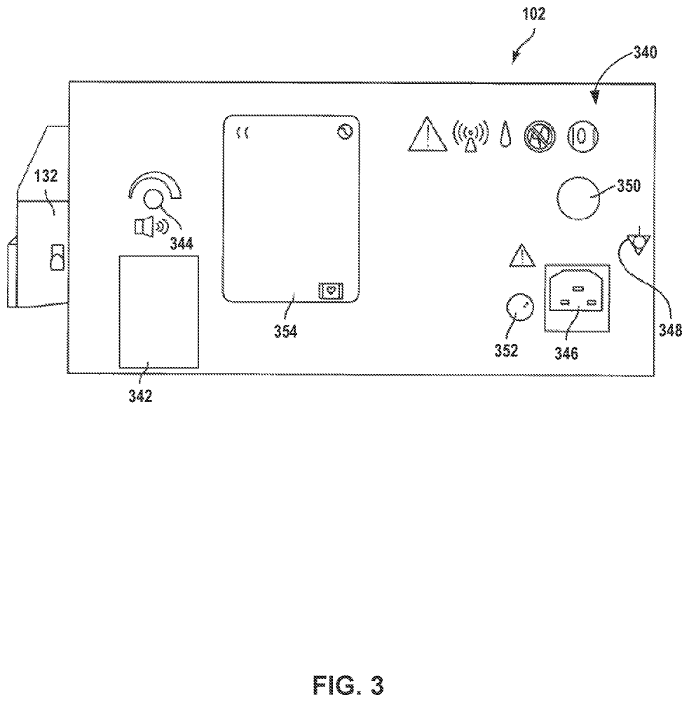

FIG. 3 shows an example rear panel 340 of the electrosurgical unit 102 of FIG. 1. The rear panel 340 of the electrosurgical unit 102 includes a speaker 342 and a volume control knob 344 to adjust the volume of the tone that will sound when the RF power is activated (“RF-power-activation tone”). The volume of the RF-power-activation tone is increased by turning the knob 344 clockwise, and decreased by turning the knob 344 counterclockwise. However, the electrosurgical unit 102 prevents this tone from being completely silenced for safety considerations.

Rear panel 340 of electrosurgical unit 102 also includes a power cord receptacle 346 used to connect the main power cord to the electrosurgical unit 102 and an equipotential grounding lug connector 348 used to connect the electrosurgical unit 102 to earth-ground using a suitable cable. The rear panel 340 also includes a removable cap 350 for the installation of a bipolar footswitch socket connectable to an internal footswitch circuit of electrosurgical unit 102 so that the RF power may be activated by a footswitch in addition to handswitch 138 of handheld device 106. Additionally, the rear panel 340 also includes a fuse drawer 352 that retains two or more extra fuses consistent with the line voltage. Finally, the rear panel 340 includes a name plate 354 which may provide information such as the model number, serial number, nominal line voltages, frequency, current and fuse rating information of the electrosurgical unit 102.

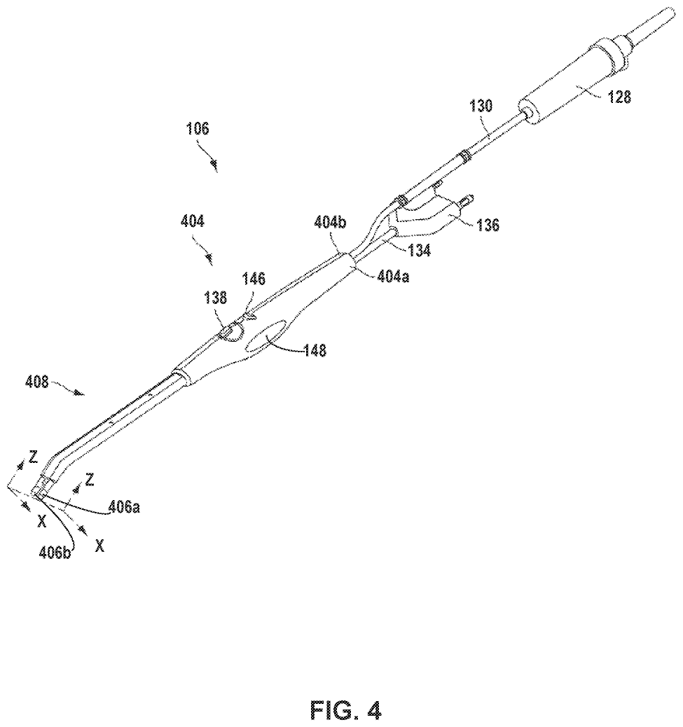

Electrosurgical unit 102 is particularly configured for use with bipolar electrosurgical devices, such as handheld device 106 of FIG. 1. With bipolar devices, an alternating-current (AC) electrical circuit is created between two electrical poles (“electrodes”) of the device. FIG. 4 is a perspective view of an exemplary bipolar electrosurgical device 106 that may be used in conjunction with electrosurgical unit 102.

As shown in FIG. 4, exemplary bipolar device 106 includes a proximal handle 404 having mating lateral handle portions 404a, 404b. Handle 404 is preferably made of a sterilizable, rigid, non-conductive material, such as a polymer (e.g., polycarbonate). Also, handle 404 is preferably configured slender, along with the rest of device 106, to facilitate a user of device 106 to hold and manipulate device 106 in a manner similar to a writing utensil. Device 106 also includes an electrical cable 134 which is connectable to electrosurgical unit 102 and flexible fluid delivery tubing 130 which is connectable to surgical-fluid source 104 (FIG. 1), preferably via a spike located at the end of drip chamber 128, which respectively provide radio-frequency energy and surgical fluid 126 to electrodes 406a, 406b.

Retained at, and connected to, the distal end of shaft 408 are two laterally and spatially separated (by empty space) contact elements including electrodes 406a, 406b which, in some examples, are configured as mirror images in size and shape, and may have a distal end with a surface devoid of edges (to provide a uniform current density) to treat tissue without cutting. Electrodes 406a, 406b are formed from an electrically conductive metal, such as stainless steel, titanium, gold, silver, and/or platinum.

In some examples, the longitudinal (e.g., distal-to-proximal) axes “Z” (FIG. 4) of electrodes 406a, 406b may be separated center-to-center (“CC”) by about 6.0 mm. As a result, when electrodes 406 have a diameter of about 3.5 mm, the actual spatial gap separation (“GS”) between electrodes 406a, 406b is about 2.5 mm.

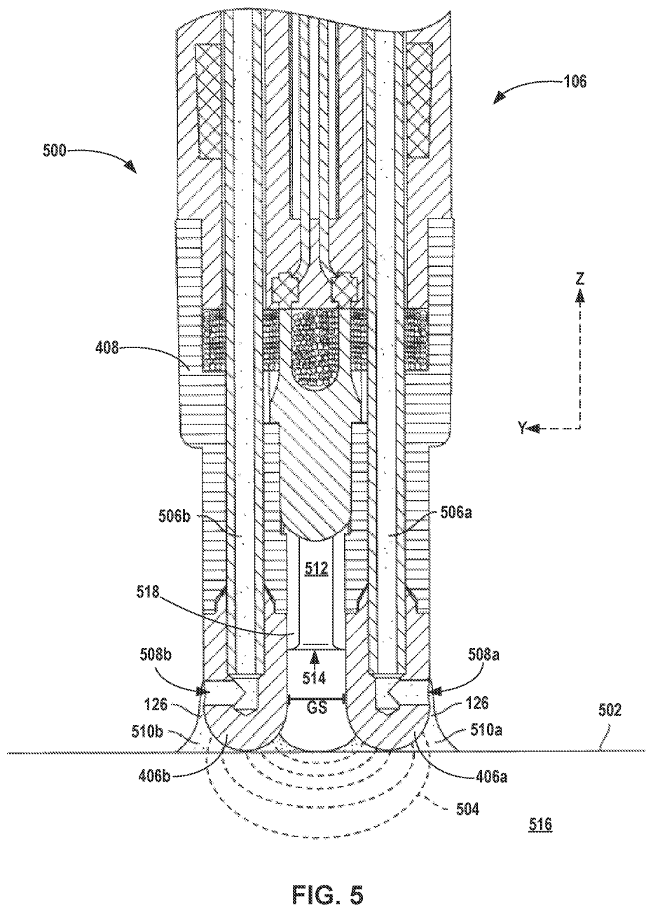

FIGS. 5-7 illustrate an example distal portion 500 of electrosurgical device 106 of FIGS. 1 and 4. As shown in FIGS. 5-7, electrodes 406a, 406b are preferably configured to slide across a surface 502 of a target tissue 516 in the presence of the radio-frequency energy 504 from electrosurgical unit 102 and the surgical fluid 126 from the fluid source 104. In some examples (but not all examples), electrodes 406a, 406b each have a domed distal shape which provides a smooth, blunt contour outer surface, e.g., which is neither pointed nor sharp.

In the example shown in FIG. 5 (but not all examples), electrodes 406a, 406b define respective inner fluid-irrigation lumens 506a, 506b, and provide surgical-fluid irrigation ports 508a, 508b for irrigation of surgical fluid 126 onto target tissue 516. Thus, during use of device 106, surgical fluid 126 from fluid source 104 (FIG. 1) is communicated through a lumen of fluid-delivery tubing 130, after which it flows through the lumens 506a, 506b where it thereafter exits device 106 from irrigation ports 508a, 508b onto electrodes 406a, 406b and target tissue 516. In the particular examples shown in FIGS. 5-7, irrigation ports 506a, 506b are located on outer-lateral portions of electrodes 406a, 406b, such that electrosurgical device 106 releases or delivers surgical fluid 126 in an outward-radial direction (e.g., along the “Y” axis).

As shown in FIG. 5, one way in which device 106 may be used is with the longitudinal (e.g., distal-to-proximal) “Z” axis of electrodes 406a, 406b vertically oriented, and the spherical distal surfaces of electrodes 406a, 406b laterally spaced (e.g., along the “Y” axis) adjacent the surface 502 of tissue 516. Electrodes 406a, 406b are connected to electrosurgical unit 102 (FIG. 1) to provide RF electrical power and form an alternating-current (“AC”) electrical field 504 in tissue 516 located between electrodes 406a and 406b. In the presence of alternating current, the electrodes 406a, 406b alternate polarity between positive and negative charges with current flowing from the positive to negative charge. Without being bound to a particular theory, a resulting heating of the target tissue 516 is performed by electrical resistance heating.

Surgical fluid 126, in addition to providing an electrical coupling between the device 106 and tissue 516, lubricates surface 502 of tissue 516 and facilitates the movement of electrodes 406a, 406b across surface 502 of tissue 516. During movement of electrodes 406a, 406b, electrodes 406a, 406b typically slide across the surface 502 of tissue 516. Typically the user of device 106 slides electrodes 406a, 406b across surface 502 of tissue 516 back-and-forth with a “painting” motion while using surgical fluid 126 as, among other things, a lubricating coating. Preferably the thickness of the fluid 126 between the distal end surfaces of electrodes 406a, 406b and surface 502 of tissue 516 at the outer edge of irrigation lumens 506 (e.g., at irrigation ports 508a, 508b, respectively) is about 0.05 mm to about 1.5 mm. Also, in certain examples, the distal-most tips of electrodes 406a, 406b may contact surface 502 of tissue 516 without any surgical fluid 126 therebetween.

As shown in FIG. 5, fluid couplings 510a, 510b include discrete, localized webs of surgical fluid 126, and more specifically, include triangular-shaped webs or bead portions providing a film of fluid 126 between tissue surface 500 and electrodes 406a, 406b. When the user of electrosurgical device 106 places electrodes 406a, 406b at a target-tissue treatment site 516 and moves electrodes 406a, 406b across the tissue surface 502, surgical fluid 126 is expelled from irrigation ports 508a, 508b and onto the tissue surface 502 in the form of surgical-fluid couplings 510a, 510b. Around the same time, electrodes 406 deliver and receive RF electrical energy, shown by electrical field lines 504, to tissue 516 via fluid couplings 510a, 510b.

In order to better maintain fluid couplings 510a, 510b as separate, discrete fluid couplings during use of electrosurgical device 106, having a gap separation “GS” between electrodes 406a, 406b of at least about 2.0 mm in combination with the positioning of irrigation ports 508a, 508b has been found to reduce undesirable merging of surgical-fluid couplings 510.

As best shown in FIG. 5, the arrangement of irrigation ports 508 defined by outer-lateral portions of electrodes 406 helps expel surgical fluid 126 onto the electrodes 406a, 406b solely at locations remote from other electrode-surface portions facing each other. More particularly, irrigation port 508a expels surgical fluid 126 onto electrode 406a at an electrode location remote from the inner-lateral surface portion of electrode 406a facing electrode 406b, and irrigation port 508b expels surgical fluid 126 onto the electrode 406b at an electrode location remote from the inner-lateral surface portion of electrode 406b facing electrode 406a.

In accordance with techniques of this disclosure, handheld device 106 is configured to both irrigate (e.g., deliver, release, or disperse, via irrigation ports 508) surgical fluid 126, and also aspirate residual surgical fluid 126. For instance, as shown in FIGS. 5-7, handheld device 106 further defines a fluid-aspiration tube 518 defining an inner fluid-aspiration lumen 512 (or “aspiration channel 512”), distally terminating in a fluid-aspiration port 514. Fluid-aspiration tube 518 is fluidically coupled, via fluid-aspiration lumen 512, to a discharge reservoir 144 (FIG. 1) configured to receive aspirated surgical fluid. Fluid-aspiration tube 518 is operatively coupled (e.g., via aspiration tubing 140 of FIG. 1), to an aspiration source 144, such as a vacuum, pump, or other suitable suction source.

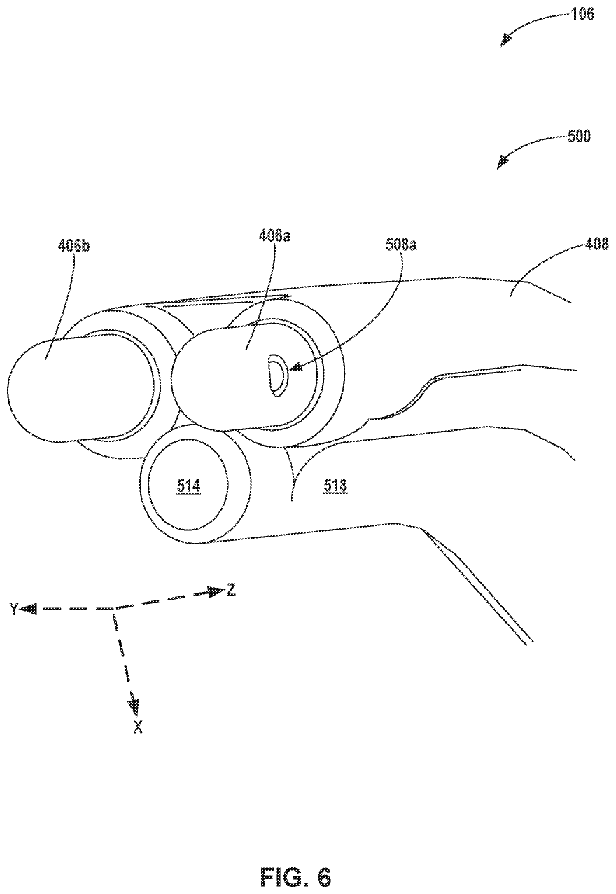

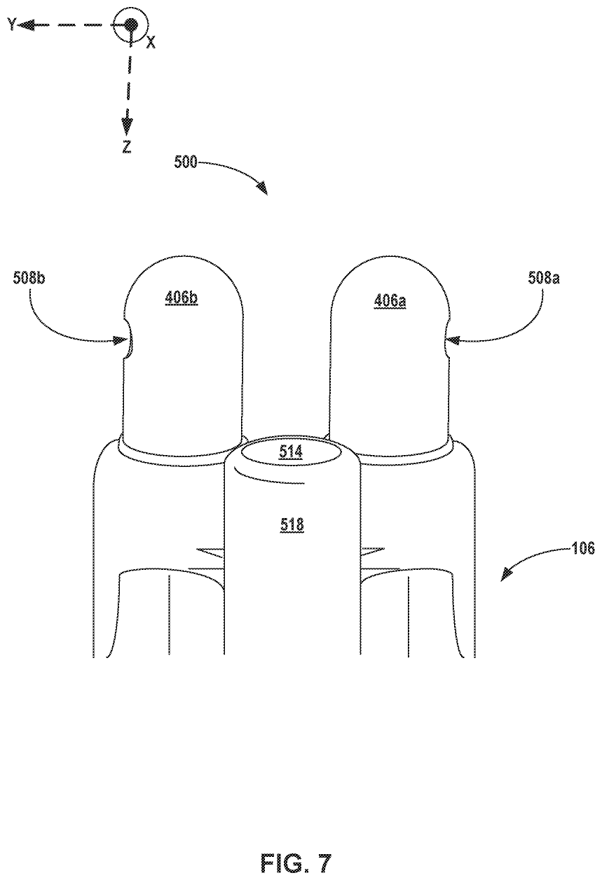

FIGS. 6 and 7 are a perspective view and an under-side view, respectively, of the example distal portion 500 of handheld electrosurgical device 106 of FIG. 5. As illustrated in FIGS. 6 and 7, in some examples, handheld electrosurgical device 106 can include a designated aspiration tube 518 that defines aspiration lumen 512 (FIG. 5) and aspiration port 514. That is, aspiration tube 518 can be physically distinguishable from, but rigidly coupled to and/or integrated with, electrodes 406a, 406b, which may extend distally from a distal-most end of an elongated shaft 408. For instance, in the particular example shown in FIGS. 5-7, aspiration tube 518 is disposed laterally between electrodes 406a, 406b along lateral axis “Y,” but slightly below electrodes 406a, 406b along vertical axis “X.” Additionally, in some examples, but not all examples, aspiration port 514 (e.g., a distal mouth of aspiration tube 518) can be positioned slightly proximally from distal-most ends of electrodes 406a, 406b (e.g., along distal-to-proximal axis “Z”). In other examples, distal-most ends of electrodes 406a, 406b and aspiration tube 518 can be approximately aligned along the “Z” axis.

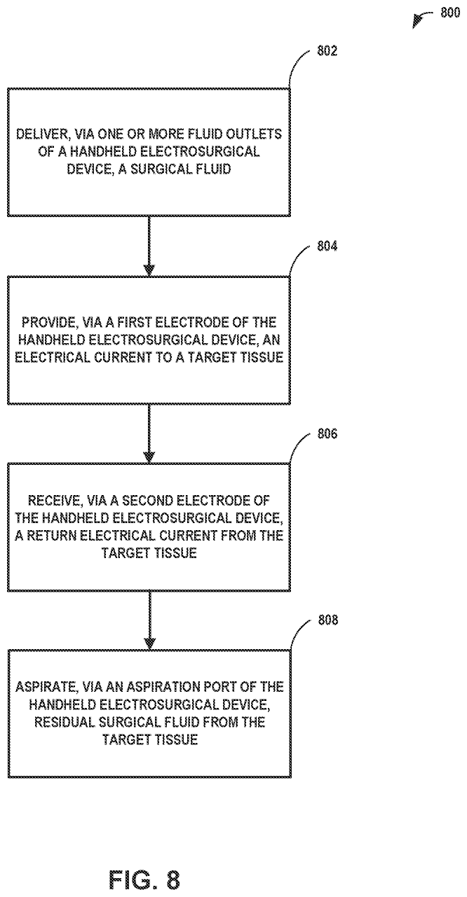

FIG. 8 is a flowchart 800 illustrating a technique for performing electrosurgery, in accordance with techniques of this disclosure. The operations of FIG. 8 are applicable to any or all of the examples of electrosurgical device 106 as shown and described herein.

At step 802, a clinician actuates a first user-input mechanism 146 of a handheld electrosurgical device 106 to deploy a surgical fluid 126, such as saline, from first and second irrigation ports 508a, 508b defined by outer-lateral portions of a pair of electrodes 406a, 406b, respectively.

At steps 804 and 806, the clinician actuates a second user-input mechanism 138 to, in the presence of the surgical fluid 126, pass an electrical current from the first electrode 406a, through a target tissue 516, and back into the second electrode 406b of the device 106, in order to seal, coagulate, etc., the target tissue 516, as appropriate.

At step 808, the clinician actuates a third user-input mechanism 148 of the handheld electrosurgical device 106 to enable a suction source 144 configured to aspirate, via one or more aspiration ports 514 defined by an aspiration tube 518 of the electrosurgical device 106, any residual surgical fluid 126, ablated tissue, or other undesired matter, from the target treatment site 516.

It should be understood that individual operations of the techniques of this disclosure may be performed in any order or simultaneously, as long as the technique remains functional for the desired outcome or result.

Examples of the present disclosure can be applied to electrosurgical devices that have additional functionality, such as providing fluid irrigation to, or fluid aspiration from, the target treatment site. In some such examples, the electrosurgical device can include conduits, ports, or passageways and be connected to a source of fluid and/or pump. Providing aspiration concurrently with electrical energy to tissue advantageously allows for aspiration of debris and/or tissues cut by the electrodes. Additional actuators may be included on the handpiece to control a flow of the fluid or suction.

Various examples of systems, devices, and techniques have been described herein. These examples are given only by way of example and are not intended to limit the scope of the claimed inventions. It should be appreciated, moreover, that the various features of the examples that have been described may be combined in various ways to produce numerous additional examples. Moreover, while various materials, dimensions, shapes, configurations and locations, etc., may have been described for use with disclosed examples, others besides those disclosed may be utilized without exceeding the scope of the claimed inventions.

Although a dependent claim may refer in the claims to a specific combination with one or more other claims, other examples can also include a combination of the dependent claim with the subject matter of each other dependent claim or a combination of one or more features with other dependent or independent claims. Such combinations are proposed herein unless it is explicitly stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. An electrosurgical device comprising:
a proximal portion comprising an electrical connector configured to electrically couple to a generator configured to provide electrical energy; and
a distal portion comprising:
a first electrode extending distally from an elongated shaft, wherein the first electrode is configured to provide a delivered electrical current to a target treatment site within a patient, and wherein the first electrode defines a first irrigation port configured to release a surgical fluid into the target treatment site; and
a second electrode extending distally from the elongated shaft, wherein the second electrode is configured to receive a return electrical current from the target treatment site, and wherein the second electrode defines a second irrigation port configured to release the surgical fluid into the target treatment site;
wherein the distal portion of the electrosurgical device defines at least one aspiration port configured to proximally aspirate the surgical fluid from the target treatment site.

2. The electrosurgical device of claim 1, further comprising an aspiration tube coupled to the elongated shaft, the aspiration tube defining the aspiration port.

3. The electrosurgical device of claim 2, wherein a distal portion of the aspiration tube is disposed between the first electrode and the second electrode along a lateral axis of the electrosurgical device, and disposed below the first electrode and the second electrode along a vertical axis of the electrosurgical device.

4. The electrosurgical device of claim 1, wherein a first outer lateral portion of the first electrode defines the first irrigation port and wherein a second outer lateral portion of the second electrode defines the second irrigation port, such that the electrosurgical device is configured to release the surgical fluid in an outward-radial direction relative to a longitudinal axis of the electrosurgical device.

5. The electrosurgical device of claim 1, wherein the surgical fluid comprises saline.

6. A method of performing electrosurgery, the method comprising:
delivering, via a first irrigation port defined by a first electrode of a distal portion of an electrosurgical device, and via a second irrigation port defined by a second electrode of the distal portion of the electrosurgical device, a surgical fluid into a target treatment site within a patient;
providing, via the first electrode, a delivered electrical current to the target treatment site;
receiving, via the second electrode, a return electrical current from the target treatment site; and
aspirating, via an aspiration port defined by the distal portion of the electrosurgical device, the surgical fluid from the target treatment site.

7. The method of claim 6, wherein aspirating the surgical fluid comprises aspirating the surgical fluid via the aspiration port of an aspiration tube coupled to first and second electrodes.

8. The method of claim 7, wherein a distal portion of the aspiration tube is disposed between the first electrode and the second electrode along a lateral axis of the electrosurgical device, and disposed below the first electrode and the second electrode along a vertical axis of the electrosurgical device.

9. The method of claim 6, wherein a first outer lateral portion of the first electrode defines the first irrigation port and wherein a second outer lateral portion of the second electrode defines the second irrigation port, such that the electrosurgical device is configured to deliver the surgical fluid in an outward-radial direction relative to a longitudinal axis of the electrosurgical device.

10. The method of claim 6, wherein delivering the surgical fluid comprises delivering saline, and wherein aspirating the surgical fluid comprises aspirating the saline.

11. A medical system comprising:
a generator configured to provide electrical energy; and
a device comprising:
a proximal portion comprising an electrical connector configured to electrically couple to the generator; and
a distal portion comprising:
a first electrode extending distally from an elongated shaft, wherein the first electrode is configured to provide a delivered electrical current to a target treatment site within a patient, and wherein the first electrode defines a first irrigation port configured to release a surgical fluid into the target treatment site; and
a second electrode extending distally from the elongated shaft, wherein the second electrode is configured to receive a return electrical current from the target treatment site, and wherein the second electrode defines a second irrigation port configured to release the surgical fluid into the target treatment site;
wherein the distal portion of the electrosurgical device defines at least one aspiration port configured to proximally aspirate the surgical fluid from the target treatment site.

12. The medical system of claim 11, wherein the electrosurgical device further comprising an aspiration tube coupled to the elongated shaft, the aspiration tube defining the aspiration port.

13. The medical system of claim 12, wherein a distal portion of the aspiration tube is disposed between the first electrode and the second electrode along a lateral axis of the electrosurgical device, and disposed below the first electrode and the second electrode along a vertical axis of the electrosurgical device.

14. The medical system of claim 11, wherein a first outer lateral portion of the first electrode defines the first irrigation port and wherein a second outer lateral portion of the second electrode defines the second irrigation port, such that the electrosurgical device is configured to release the surgical fluid in an outward-radial direction relative to a longitudinal axis of the electrosurgical device.

15. The medical system of claim 11, wherein the surgical fluid comprises saline.
